(19) United States
(12) Patent Application Publication
(10) Pub. No.: US 2010/0106039 A1
(43) Pub. Date: Apr. 29, 2010

(54) Unknown

(76) Inventors: Klaus Abraham-Fuchs, Erlangen (DE); Karsten Hiltawsky, Schwerte (DE); Oliver Hornung, Furth (DE); Thomas Krüger-Sundhaus, Pommersfelden (DE); Erhard Magori, Feldkirchen (DE); Peter Paulicka, Erlangen (DE); Roland Pohle, Herdweg (DE); Oliver Von Sicard, Munich (DE)

Correspondence Address: HARNESS, DICKEY & PIERCE, P.L.C., P.O.BOX 8910, RESTON, VA, 20195, US

(21) Appl. No.: 12/585,934

(22) Filed: Sep. 29, 2009

(30) Foreign Application Priority Data
Sep. 30, 2008 (DE) 10 2008 049 768.1

(51) Int. Cl. A61B 5/08

(52) U.S. Cl. 600/532

(57) ABSTRACT

Example embodiments relate to a device for measuring nitrogen monoxide in exhaled air by a gas sensor unit with at least one gas sensor. A device and/or method for oxidation of nitrogen monoxide to nitrogen dioxide may be included. The device and/or method for oxidation of nitrogen monoxide to nitrogen dioxide includes a non-consuming catalyst for catalyzing the oxidation of nitrogen monoxide to nitrogen dioxide.

### PRIORITY STATEMENT

The present application hereby claims priority under 35 U.S.C. §119 to German patent application number DE 10 2008 049 768.1, filed Sep. 30, 2008, the entire contents of which are hereby incorporated herein by reference.

### FIELD

At least one example embodiment of the present application generally relates to an arrangement for measuring the concentration of nitrogen oxide (NO) in respiratory gas and/or to a method for measuring the concentration of NO.

### BACKGROUND

Nitrogen oxide (nitrogen monoxide, NO) is a marker that is released continuously from cells of the airways into the flow of respiratory gas and that represents an important marker for diagnosing asthma and for optimizing the treatment of asthma. Affecting about 5% of adults and about 20% of children in the developed industrialized nations, asthma is one of the most commonly occurring diseases. In inflammatory processes of the airways, e.g. asthma, increased NO concentrations of 40 ppb (parts per billion), or more, occur in the exhaled air. Imminent asthma attacks can already be detected much earlier by an increase in the NO content of the exhaled air than is possible by a lung function test. Consequently, measuring NO in exhaled air is a preferred method for diagnosing and for monitoring the treatment of asthma and other inflammatory diseases of the airways.

Low-cost NO sensors having the required sensitivity in the ppb range have not hitherto been available on the market. A newly developed NO₂ sensor based on suspended-gate FET technology meets the stated requirements. However, a sensor of this kind has to be provided with an upstream conversion module for converting the NO in the respiratory gas to NO₂, which can be detected by the sensor. A conversion module of this kind should ideally last for several months or even years, should be inexpensive, and should convert NO to NO₂ at a very high and constant rate of conversion.

This purpose was hitherto served by oxidizing agents which, for example, were provided in an upstream chamber and through which the respiratory gas was conveyed. Possible oxidizing agents, described in DE 101 212 62 A1, for example, are permanganate salts and perchlorate salts. A disadvantage of this solution is that the oxidizing agent itself is consumed, and the conversion module thus eventually loses its ability to convert NO to NO₂.

### SUMMARY

At least one example embodiment makes available an arrangement that measures NO in respiratory air and that is inexpensive and reusable.

According to example embodiments, it is proposed to use a conversion module that is not consumed and/or can continuously be regenerated through the use of a non-consuming oxidation catalyst.

At least some example embodiments provide a device for measuring nitrogen monoxide in exhaled air by a gas sensor unit with at least one gas sensor, wherein a means for oxidation of nitrogen monoxide to nitrogen dioxide is provided, wherein the means for oxidation of nitrogen monoxide to nitrogen dioxide includes a non-consuming catalyst for catalyzing the oxidation of nitrogen monoxide to nitrogen dioxide.

According to an example embodiment, the catalyst is a thermally activatable catalyst and the device may comprise a heater.

According to another example embodiment, the oxidation catalyst is a photochemical catalyst (photocatalyst) and the device may comprise a light source.

According to another example embodiment, the device includes a means for generating activated oxygen species, in particular, a means for generating ozone or singlet oxygen.

The catalyst may be active at a temperature of less than 300° C., preferably less than 250° C., particularly preferably less than 200° C. The catalyst, at a temperature of less than 300° C., preferably less than 250° C., particularly preferably less than 200° C., should reach at least 80% of the reaction rate that it reaches at its temperature optimum.

This is particularly advantageous since complete, or more complete, conversion of NO to NO₂ is possible at lower temperatures (in this connection see also FIG. 2).

The means for oxidation of nitrogen monoxide to nitrogen dioxide may be as close as possible to the sensor, for example at the inlet opening to the measuring chamber, or integrated in the measuring chamber itself, such that the converted gas can be measured as directly as possible.

In another example embodiment, the means for oxidation of nitrogen monoxide to nitrogen dioxide is integrated in the sensor element. This can be achieved by a layered structure (e.g., catalyst layer on sensor layer) or by a monolithic structure (e.g., catalyst dispersed in sensor layer).

In another example embodiment, a calibrating gas of defined NO concentration acts on a gas analysis appliance (e.g., at selectable time intervals) for quality control or calibration. This calibration procedure can also be used to measure the rate of action of the conversion module and, if the rate of action falls, to activate the regeneration.

According to at least one example embodiment, the gas sensor unit includes a NO₂-sensitive field-effect transistor sensor (FET sensor).

The conversion of nitrogen monoxide to nitrogen dioxide takes place according to the following reaction equation:

2NO+O₂ [symbol] 2NO₂, ΔH=−114 kJ/mol

Since the reaction enthalpy is negative, the reaction takes place in the direction of conversion to NO₂, in other words it only has to be made possible by a catalyst. In this connection, it will be noted that most of the oxygen present in the ambient air is present also in exhaled air, since only a small part thereof is consumed during breathing.

The respiratory air of humans may also contain other metabolic byproducts with a reducing action (e.g., ketones or alcohols). If a sensor element is used that is not selective to this, there is a danger of the measurement being disturbed. A further advantage is that, when using an active oxidation catalyst, these byproducts are oxidized to undetected CO₂ and H₂O and the selectivity of the nitrogen oxide measurement is thus increased.

### BRIEF DESCRIPTION OF THE DRAWINGS

Example embodiments will be more clearly understood from the following detailed description taken in conjunction with the accompanying drawings. FIGS. 1-4 represent non-limiting, example embodiments as described herein.

FIG. 1 shows a schematic view of an example embodiment of an device for measuring NO; and

FIG. 2 shows a graph illustrating the rate of conversion of NO to NO₂ as a function of temperature.

### DETAILED DESCRIPTION OF THE EXAMPLE EMBODIMENTS

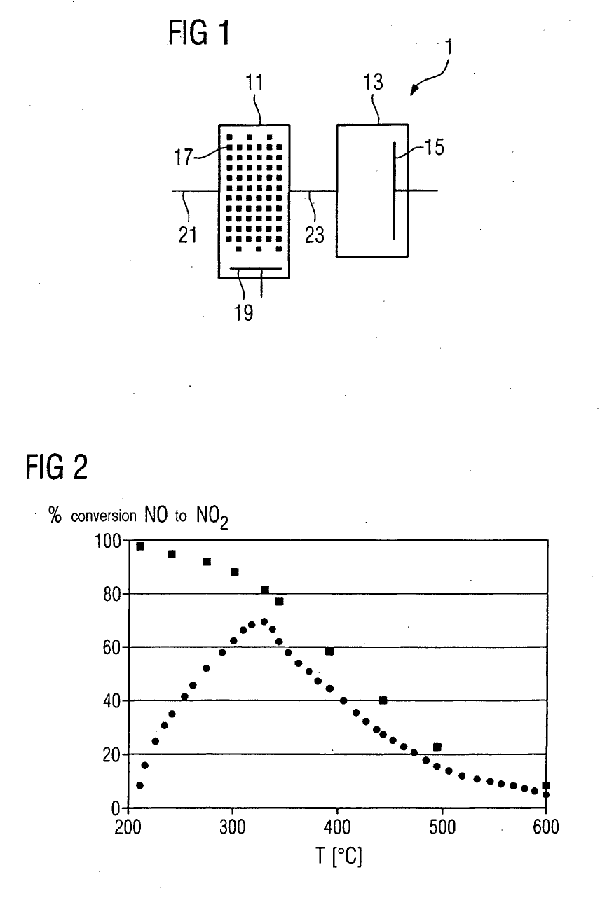

FIG. 1 shows by way of example, and schematically, an example embodiment of a device 1 for measuring NO with a conversion module 11 and a gas sensor unit 13. By way of an admission line 21, exhaled air is conveyed into the conversion module 11, in which an oxidation catalyst 17 is provided. In the case of a thermally activatable oxidation catalyst, a heater 19 can additionally be provided, e.g., in the form of an electrically resistive heater. After the catalytic conversion, the exhaled air is conveyed through a line 23 into the gas sensor unit 13, in which a NO₂-sensitive gas sensor 15 is provided.

As gas sensor 15, it is possible, for example, to use a NO₂-sensitive sensor based on a transistor. When using nitrogen oxide detection according to the principle of work function measurement, various field-effect transistors are known in which the gas-sensitive layer is formed as a gate electrode. This gate electrode can be separated by an air gap from a channel area of the field-effect transistor. The basis for a detecting measurement signal is the change in potential between a gate and the channel area (ΔV_{G}). German patent applications No. 198 14 857.7 and No. 199 56 806.5, for example, describe hybrid flip-chip constructions of gas sensors that are designed as CMOS transistors. A gas sensor can additionally be equipped with two field-effect transistors whose control behavior is balanced by approximately identical air gaps between channel area and gate electrode and whose sensor layers can be read out separately. German patent application No. 199 56 744.1 describes how the distance between the gate electrode and channel area of a field-effect transistor can be reproduced by extremely precise spacers. In another configuration, gas-sensitive material in porous form is applied to the channel area or to the gate.

Gas-sensitive layers for use in an SG-FET (suspended-gate field-effect transistor) can be porphin dyes, e.g., phthalocyanines, with the central atom copper or lead. At sensor temperatures of between 50° and 120° C., nitrogen oxide sensitivities can be detected down into the low ppb range. The detection is directed as usual at nitrogen dioxide.

Other materials suitable for use in gas-sensitive field-effect transistors and as gas-sensitive layers for detection of nitrogen oxide, in particular nitrogen dioxide, are finely crystalline metal oxides at temperatures of between 80° and 150° C. These metal oxides can be SnO₂, WO₃ and In₂O₃, while salts from the carbonate system, such as barium carbonate, or polymers, such as polysiloxanes, are also conceivable.

Catalysts based on a very fine dispersion of a noble metal catalyst, e.g. platinum, rhodium or palladium, are suitable for carrying out the conversion with the best possible efficiency at low temperature. These include, for example, platinum black, a so-called “supported catalyst”, that is to say as catalyst dispersion that is applied to a support body. Suitable support bodies are, among others, open-pore metal oxide or ceramic support bodies through which gas can flow and extruded non-porous oxide shaped bodies with an integrated channel structure in which the gas flows through the channels in order to allow maximum contact with the catalyst surface. Alternatively, a sufficiently thermally stable polymer can also be provided as support for the catalyst. Suitable materials are, for example, PMMA, PDMA or polyimides. For this purpose, a shaped body is produced by microtechnology processes and is provided with micro-channels by current.

It is also possible to provide a fine net or a fiber-like structure of small catalytically active wires or fibers (e.g., platinum). Since such structures are also electrically conductive, they can be used at the same time for heating.

For a high degree of oxidation with respect to NO, oxides of rare earth metals and/or of transition metals, for example, can be used as a catalyst material, as a catalyst component or as a coating component. One or more oxides from the group including V₂O₅, Cr₂O₃, Mn₂O₃, MnO₂, Mn₃O₄, Fe₂O₃, Fe₃O₄, CoO, CO₃O₄,

NiO, NiO₂, Ni₂O₃, CeO₂ and Ce₂O₃ may be used. These catalysts can also be provided as an additive in noble metals catalysts.

Oxides of rare earth metals and/or of transition metals used as catalyst material or catalyst additive have the advantage that these catalysts are active at lower temperatures of below 250°. This advantage is also provided by photocatalysts.

The graph in FIG. 2 illustrates the conversion of nitrogen monoxide to nitrogen dioxide as a function of temperature. At temperatures of >200° C., there is an almost complete conversion to NO₂. As temperatures rise, the balance shifts in the direction of NO. This is shown by blocks in the figure. The graph also shows schematically and without dimensions the conversion rate (shown by circles) by the catalyst (in this example Pt) as a function of temperature. Although the chemical equilibrium is strongly on the side of conversion to NO₂ at low temperatures, the reaction speed is too slow. Through use of a suitable catalyst, the conversion can take place at temperatures of 150° C. to 250° C., in which range a complete or almost complete conversion of NO to NO₂ can take place.

According to another example embodiment, the catalyst is heated to a higher temperature (300-450° C.) shortly before the conversion, with reactive oxygen species forming on the surface. The catalyst is then cooled and the conversion carried out, the lower temperature again ensuring a complete conversion to NO₂. A corresponding control device is provided which heats the catalyst shortly before the measurement. This control device may have a signal device which, after the catalyst has been heated and thereafter cooled to the conversion temperature, signals that the measurement can now take place. The control device may also include a temperature sensor.

To lower the required temperatures, the catalytic action can be further improved by addition of catalytic promoters, e.g., metals such as rhodium, rhenium, osmium or oxides such as cerium oxide, iron oxide, hafnium oxide or lanthanum oxide.

Alternatively, the oxidation of the nitrogen monoxide can also be carried out using a photocatalyst. In this embodiment, no heating is required. However, light energy may be supplied. For this purpose, a light source is provided. UV light is usually provided, e.g. by a UV-LED or a UV lamp. TiO₂ may be provided as the photocatalyst. TiO₂ is used in the reactive crystal structure anatase in order also to obtain a high reactivity by means of a correspondingly small particle size of the catalyst (e.g., less than 50 nm).

The catalysts generally have a limited conversion capacity. To optimize the catalyst volume and the required temperature, a structure is advantageously provided in which only some of the gas stream is guided through the catalyst. The rest of the gas stream is not fed to the sensor and leaves the appliance. This permits a longer dwell time in the catalyst and therefore an improved conversion.

It will also be noted that catalysts have, on their surface, a certain storage capacity for nitrogen oxides, and this can lead to inaccurate measurements. Consequently, the structure may be configured in such a way that, before the measurement, the catalyst is flushed for a sufficiently long time (e.g. 2 seconds) with respiratory gas so as to ensure that no nitrogen oxide is subsequently lost. After the measurement, the catalyst is flushed, maybe for at least the same length of time, with air that is free of nitrogen oxide, so as to avoid nitrogen oxides being entrained into the next measurement cycle. A suitable control arrangement may be provided which measures the flow of gas and which signals that the catalyst has been flushed with respiratory air for a sufficient length of time before the measurement or has been flushed with air free of nitrogen oxide after the measurement interval.

The conversion of NO to NO₂ can also be assisted by generating active oxygen species. This can be done in particular by ozone (O₃) which is generated, for example, by an integrated UV light source or by a miniaturized electric discharge (corona type). However, for complete reaction of the nitrogen oxide with the generated ozone, a catalytically active surface is also needed. Another example of an activated oxygen species is singlet oxygen, a highly reactive variant of the O₂ molecule normally present in the triplet state. Singlet oxygen reacts immediately with other gases, and the generation of singlet oxygen takes place photochemically by light irradiation of suitable dyes, e.g. methylene blue, eosin, Bengal pink or phthalocyanine, e.g. with light in the near infrared range from a suitable LED. A wavelength of 850-1250 nm is normally used.

The conversion module may be provided as close as possible to the sensor, e.g. at the inlet opening to the measuring chamber or integrated in the measuring chamber itself, such that the converted gas can be measured as directly as possible.

According to another example embodiment, the conversion module is integrated in the sensor element itself (hybrid or monolithic). This can be achieved by a two-layer structure (a catalyst layer on the sensor layer) or by a monolithic structure (the sensor surface is located on the same support body and is mixed homogeneously or heterogeneously with the catalytically active material). A heater is integrated on the surface or in the material of the conversion module and regenerates the oxidative capacity of the module. The heater can be started up automatically, being controlled, for example, by measuring the operating hours or by measuring the flow of gas through the module. In another example embodiment, a calibrating gas of defined NO concentration acts on the gas analysis device at selectable time intervals for quality control or calibration. This calibration procedure can also be used to measure the rate of action of the conversion module and, if the rate of action falls, to activate the regeneration.

Important advantages of the overall system are that a non-invasive measurement method is used. The measurements can be repeated in large numbers and can thus also be used for monitoring of treatments, for diagnosis of asthma in children, for early detection of asthma or for preventative medical measures. Through the use of non-consuming catalysts, the device requires minimal maintenance and permits inexpensive measurements. The system proposed here is therefore also suitable for hospitals and medical practices.

Example embodiments being thus described, it will be obvious that the same may be varied in many ways. Such variations are not to be regarded as a departure from the spirit and scope of the present disclosure, and all such modifications as would be obvious to one skilled in the art are intended to be included within the scope of the following claims.

1. A device for measuring nitrogen monoxide in exhaled air comprising:
a gas sensor unit with at least one gas sensor; and
means for oxidation of nitrogen monoxide to nitrogen dioxide, the means for oxidation including a non-consuming catalyst for catalyzing the oxidation of nitrogen monoxide to nitrogen dioxide.

2. The device as claimed in claim 1, wherein the catalyst is a thermally activatable catalyst.

3. The device as claimed in claim 2, further comprising;
a heater.

4. The device as claimed in claim 1, wherein the catalyst is a photochemical catalyst.

5. The device as claimed in claim 4, further comprising;
a light source.

6. The device as claimed in claim 1, further comprising:
a means for generating activated oxygen species.

7. The device as claimed in claim 1 wherein the gas sensor is a NO₂-sensitive FET sensor.

8. The device as claimed in claim 1, wherein the means for oxidation of nitrogen monoxide to nitrogen dioxide is provided at an inlet opening to a measuring chamber.

9. The device as claimed in claim 1, wherein the means for oxidation of nitrogen monoxide to nitrogen dioxide is integrated in the gas sensor.

10. The device as claimed in claim 1 further comprising;
a means for applying a calibrating gas having an NO concentration on the device.

11. The device as claimed in claim 1, wherein the catalyst is active at a temperature of less than 300° C.

12. The device as claimed in claim 1, wherein the means for oxidation of nitrogen monoxide to nitrogen dioxide is provided in a measuring chamber.

13. The device as claimed in claim 11, wherein the catalyst is active at a temperature of less than 250° C.

14. The device as claimed in claim 13, wherein the catalyst is active at a temperature of less than 200° C.

15. The device as claimed in claim 1, wherein the catalyst is at least one of V₂O₅, Cr₂O₃, Mn₂O₃, MnO₂, Mn₃O₄, Fe₂O₃, Fe₃O₄, CoO, CO₃O₄, NiO, NiO₂, Ni₂O₃, CeO₂ Cr₂O₃, Mn₂O₃, MnO₂, Mn₃O₄, Fe₂O₃, Fe₃O₄, CoO, CO₃O₄, NiO, NiO₂, Ni₂O₃, CeO₂ and Ce₂O₃.

16. The device as claimed in claim 4, wherein TiO₂ is the photochemical catalyst.

17. The device as claimed in claim 16, wherein a particle size of the TiO₂ is less than fifty nm.
